(19) United States
(12) Patent Application Publication
(10) Pub. No.: US 2023/0347160 A1
(43) Pub. Date: Nov. 2, 2023

(54) PLASMA TREATMENT DEVICE

(71) Applicants: PLARIT CO.,LTD., Jeollabuk-do (KR); INNOVATION FOR CREATIVE DEVICES CO., LTD., Anseong-si Gyeonggi-do (KR); AJOU UNIVERSITY INDUSTRY-ACADEMIC COOPERATION FOUNDATION, Suwon-si Gyeonggi-do (KR)

(72) Inventors: Chul Ho KIM, Seoul (KR); Seung Ho LEE, Yongin-si (KR); Yun Sang LEE, Seoul (KR); Sung Un KANG, Suwon-si (KR); Boo Ki MIN, Ansan-si (KR)

(73) Assignees: PLARIT CO.,LTD., Jeollabuk-do (KR); INNOVATION FOR CREATIVE DEVICES CO., LTD., Anseong-si Gyeonggi-do (KR); AJOU UNIVERSITY INDUSTRY-ACADEMIC COOPERATION FOUNDATION, Suwon-si Gyeonggi-do (KR)

(21) Appl. No.: 18/344,488

(22) Filed: Jun. 29, 2023

Related U.S. Application Data
(63) Continuation of application No. PCT/KR2020/019297, filed on Dec. 31, 2020.

(51) Int. Cl. A61N 1/44; H05H 1/28

(52) U.S. Cl. CPC A61N 1/44; H05H 1/28; H05H 2245/34

(57) ABSTRACT

The present invention relates to a plasma treatment device, and more particularly, to a plasma treatment device effective in treating atopic dermatitis, which amplifies a low voltage to a high voltage within a handpiece in a plasma generating process to reduce ozone production and improve stability.

### CROSS-REFERENCE TO RELATED APPLICATIONS

The present application is a Continuation of International Patent Application No. PCT/KR2020/019297 filed on Dec. 31, 2020, the entire contents of which are incorporated herein by reference.

### TECHNICAL FIELD

The present invention relates to a plasma treatment device, and more particularly, to a plasma treatment device effective in treating atopic dermatitis, which amplifies a low voltage to a high voltage within a handpiece in a plasma generating process to reduce ozone production and improve stability.

### BACKGROUND ART

Plasma is called the fourth state of matter and refers to a mixture of negatively charged electrons and positively charged ions and neutral particles (molecules and atoms). Electrons can be easily accelerated in a variety of ways, neutral particles collide with molecules to create chemically active species, and ions create conditions in which a surface of a material to be treated may be chemically reacted to cause the active species to actively chemically react on the surface. In addition, plasma is used in a variety of industries, including semiconductor, display, energy, machinery, chemical, and bio, because the plasma can be generated at high pressures, that is, above atmospheric pressure from a high vacuum and can be discharged even in a liquid state as well as gas, making it possible to selectively use plasma generation methods suitable for various production conditions. In particular, plasma is actively utilized in bio fields, such as biomedical field, food industry, and agriculture, because plasma has excellent sterilization power by generating active species with strong oxidizing power and high reactivity. Recently, research has been conducted to fundamentally treat intractable diseases, such as atopy, dermatitis, Alzheimer's disease, Parkinson's disease, and cancer, by using active species, and it has been confirmed that plasma has excellent effects on skin regeneration.

Plasma treatment devices for the treatment of dermatitis mainly use atmospheric pressure low temperature plasma. Atmospheric pressure low temperature plasma is known to have a strong sterilization effect and cell growth stimulation effect, and as a result, skin treatment devices using atmospheric pressure low temperature plasma are increasingly being used to treat dermatitis, such as atopy, athlete's foot, eczema, and acne, as well as wounds and burns. In addition, atmospheric pressure low temperature plasma treatment devices may prevent thinning and discoloration of the skin, a side effect of using steroid anti-inflammatory drugs known as conventional dermatitis treatment and prevention materials.

However, the plasma treatment devices in the related art produce ozone, nitrogen dioxide, and nitrogen monoxide, which are harmful to humans when used for long periods of time. In particular, ozone has a fishy odor that can irritate the nose and throat, causes breathing difficulties and decreased lung function at high ozone concentrations, and causes headaches and eye redness in confined spaces.

In addition, the plasma treatment devices in the related art generate plasma by high voltage inside the main body. In this case, when the high-voltage plasma is applied through the cable to the handpiece, voltage and signal noise may be generated, thereby causing a problem in stability against the high voltage.

### DISCLOSURE

Technical Problem

The present invention is conceived in response to the background art, and has been made in an effort to provide a plasma treatment device in which ozone generation is effectively reduced when plasma is generated through a control of a flow rate of argon gas.

The present invention is conceived in response to the background art, and has been made in an effort to provide a plasma treatment device which amplifies low voltage to high voltage within a handpiece in a plasma generation process to reduce a voltage in a cable and noise from signals and improve stability to high voltage.

### Technical Solution

An exemplary embodiment of the present invention provides a plasma treatment device, including: a handpiece for generating plasma and emitting the generated plasma onto skin; a main body for supplying voltage and gas to the handpiece; and a hybrid cable connecting the main body and the handpiece, in which the handpiece amplifies a low voltage to a high voltage to generate the plasma.

Further, the handpiece may include: a plasma generating unit for generating the plasma by being supplied with the voltage and the gas; a plasma output unit for emitting the plasma onto the skin; and a handpiece tip positioned on a front surface of the plasma output unit to maintain a constant distance between the plasma output unit and the skin, and the plasma is generated by amplifying a direct current voltage of 9 V to 10 V to an alternating current voltage of 700 V to 900 V at a frequency of 18 kHz to 20 kHz.

Further, the plasma generating unit includes a dielectric including one or more of quartz, sapphire, glass, ceramic, polymer film, and polyetherimide (PEI).

Further, the plasma output unit is formed with a nozzle which emits the plasma, and the nozzle is at least one.

Further, the handpiece tip is replaceable.

Further, the main body includes: a power unit for receiving an alternating current voltage; a converter for converting the alternating current voltage to a direct current voltage; a pneumatic unit for controlling the flow rate and output of the gas; a control unit for controlling an overall system operation; and a monitor for displaying medical information.

Further, the power unit is applied with an alternating current voltage of 220V, and the converter converts the alternating current voltage to a direct current voltage of 24 V.

Further, the pneumatic unit includes: a pressure sensor for measuring a pressure of the gas; an electronic valve for controlling an output of the gas; and a flow controller for controlling a flow rate of the gas.

Further, the plasma treatment device further includes a gas supply unit for supplying the gas, in which the gas supply unit includes: a gas bombe for storing the gas; a gas tube for transporting the gas; and a gas socket connecting the main body and the gas tube, and the gas is argon gas, and a flow rate of the gas is from 4 l/m to 5 l/m.

Further, the main body includes a ventilation opening for dissipating heat within the main body.

### Advantageous Effects

According to the present invention, plasma is generated and emitted to the skin by using argon gas, thereby exhibiting an excellent effect on skin regeneration treatment and atopic dermatitis by suppressing a hypersensitive immune response, and in particular, exhibiting an excellent effect on atopic treatment by inhibiting the differentiation of Th2 cells, which are the main cause of atopic dermatitis, and inhibiting the infiltration of mast cells and eosinophils into skin tissue.

In addition, by controlling the flow rate of argon gas, ozone generated during plasma generation may be effectively reduced.

In addition, by generating plasma by amplifying low voltage to high voltage within the handpiece, high voltage does not flow in the cable connecting the main body and the handpiece, which improves the stability of the cable, reduces noise, and simplifies manufacturing.

### DESCRIPTION OF DRAWINGS

FIG. 1 is a perspective view of a plasma treatment device 1000.

FIG. 2A is a front view of the plasma treatment device 1000, FIG. 2B is a side view of the plasma treatment device 1000, and FIG. 2C is a rear view of the plasma treatment device 1000.

FIG. 3 is a block diagram of the plasma treatment device 1000.

FIG. 4A is a perspective view of the handpiece 100, FIG. 4B is a side perspective view of the handpiece 100, and FIG. 4C is an internal cross-sectional view of the handpiece 100.

FIGS. 5A and 5B are graphs illustrating the amount of ozone generated according to applied voltage of the plasma treatment device.

FIGS. 6A and 6B are graphs illustrating the amount of ozone generated according to a flow rate of argon gas of the plasma treatment device.

FIG. 7 is a graph illustrating the amount of ozone generated when plasma is generated by supplying an applied voltage of 9V and an argon gas flow rate of 51/m to a handpiece test product of the plasma treatment device.

### MODE FOR CARRYING OUT THE INVENTION

The present invention will be described in detail with reference to the accompanying drawings. Herein, the repeated description, and the detailed description for the publicly known function and configuration may be omitted so as to avoid unnecessarily obscuring the subject matter of the present invention. An exemplary embodiment of the present invention is provided to fully explain the present invention to those skilled in the art. Accordingly, shapes, sizes, and the like of the elements in the drawing may be exaggerated for clearer description.

In the entire specification, unless explicitly described to the contrary, the word “comprise” and variations such as “comprises” or “comprising” will be understood to imply the inclusion of stated elements but not the exclusion of any other elements.

Hereinafter, an exemplary embodiment is presented for helping the understanding of the present invention. However, the following exemplary embodiment is simply provided for more easy understanding of the present invention, and the contents of the present invention are not limited by the exemplary embodiment.

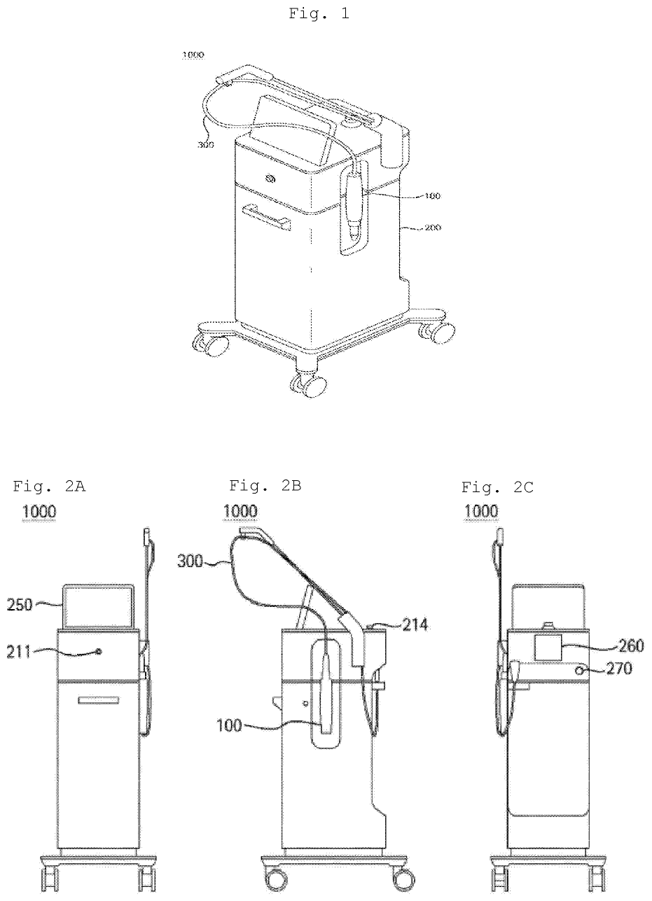

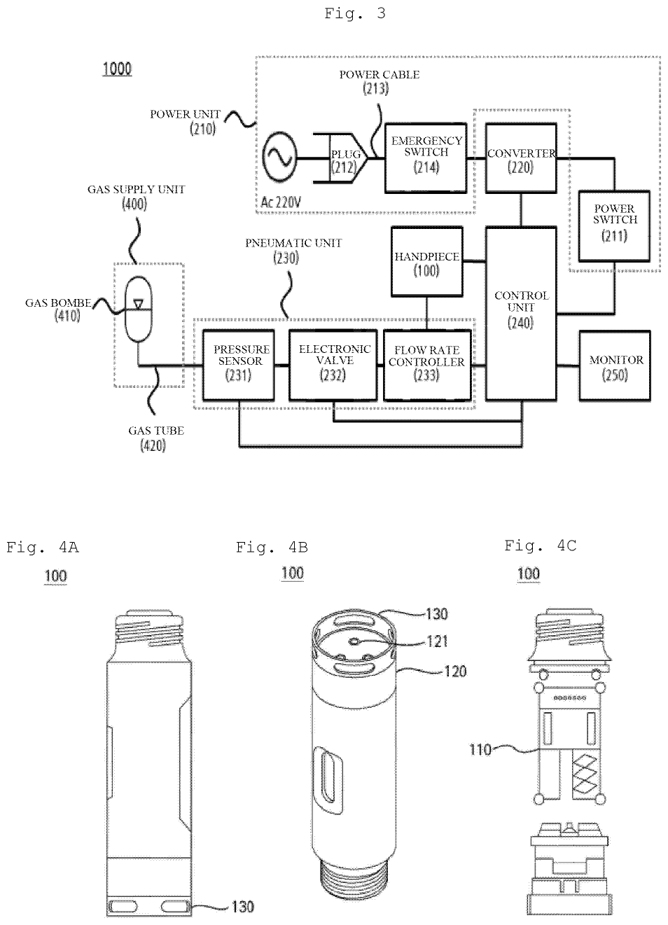

FIG. 1 is a perspective view of a plasma treatment device 1000, FIG. 2A is a front view of the plasma treatment device 1000, FIG. 2B is a side view of the plasma treatment device 1000, and FIG. 2C is a rear view of the plasma treatment device 1000, and FIG. 3 is a block diagram of the plasma treatment device 1000. Referring to FIGS. 1 to 3, a plasma treatment device 1000 according to the present disclosure may include a handpiece 100, a main body 200, and a hybrid cable 300.

The handpiece 100 according to the present invention may generate plasma and emit the generated plasma to the skin. FIG. 4A is a perspective view of the handpiece 100, FIG. 4B is a side perspective view of the handpiece 100, and FIG. 4C is an internal cross-sectional view of the handpiece 100. Referring to FIGS. 4A to 4C, the handpiece 100 may include a plasma generating unit 110, a plasma output unit 120, and a handpiece tip 130.

The plasma generating unit 110 is formed inside the handpiece 100 and may generate plasma by receiving voltage and gas from the main body 200, which will be described later. In the plasma generating unit 110, an electrode that receives voltage from the main body 200 and a ground electrode installed at a certain distance from the electrode form an electric field. In this case, when gas is injected, a glow discharge is generated by the pressure generated between the electrode and the ground electrode and the flow rate of the gas, so that plasma may be generated. Here, a dielectric of one or more of quartz, sapphire, glass, ceramic, polymer film, and polyetherimide (PEI) may be positioned between the electrode and the ground electrode to prevent the transfer of glow discharge, thereby suppressing leakage and concentration of plasma. In this case, the plasma generating unit 110 receives a direct current voltage of 9V to 10V from the main body 200. Herein, in order to generate plasma, when a frequency of 18 kHz to 20 kHz is set to supply a direct current voltage of 9V to 10V, the direct current voltage is amplified to an alternating current voltage of 700V to 900V to generate plasma. In this case, since the plasma is generated by amplifying a low voltage to a high voltage, the amount of harmful gas generated when the plasma is generated is greatly reduced so that ozone (O₃) is less than or equal to 0.05 ppm, nitrogen dioxide (NO₂) is less than or equal to 0.05 ppm, and nitrogen monoxide (NO) is less than or equal to 20 ppm, and thus the stability of the plasma treatment device 1000 may be improved. In addition, the stability may be further improved by separately configuring a harmful gas discharge unit (not illustrated) to prevent direct contact of harmful gas with the skin.

The plasma output unit 120 may emit the generated plasma onto the skin. The plasma may be emitted from a nozzle 121 formed in the plasma output unit 120. One or more nozzles 121 may be formed to output multiple plasma to improve the treatment effect.

The handpiece tip 130 may be positioned on a front surface of the plasma output unit 120 to maintain a constant distance between the plasma output unit 120 and the skin. The handpiece tip 130 is designed to be replaceable so that the handpiece tip 130 may be removed from the handpiece 100 and immediately replaced in the event of contamination or destruction. Additionally, the handpiece tip 130 may be made of a material that has been tested for biological safety due to the direct contact with the skin, and may be preferably made of polycarbonate.

The main body 200 according to the present invention may supply voltage and gas to the handpiece 100. The main body 200 may include a power unit 210, a converter 220, a pneumatic unit 230, a control unit 240, and a monitor 250.

The power unit 210 may receive an alternating current voltage to enable the plasma treatment device 1000 to operate. The power unit 210 may receive a voltage by driving a power switch 211. The voltage may receive an alternating current voltage of 220 V at 60 Hz from the outlet through a plug 212, and transmit the voltage to the converter 220 which will be described below through a power cable 213. The power unit 210 may be provided with an emergency switch 214 to check the voltage supply or the voltage of the circuit and immediately cut off the power when abnormality occurs, thereby preventing failure of the plasma treatment device 1000.

The converter 220 may convert the alternating current voltage of 220 V received from the power unit 210 to a direct current voltage of 24 V to supply power to the control unit 240 described later.

The pneumatic unit 230 may control a flow rate and output of gas, and may include a pressure sensor 231, an electronic valve 232, and a flow controller 233. The pressure sensor 231 may measure the pressure of the gas to monitor the amount of gas remaining in a gas bombe 410 which will be described later. The electronic valve 232 may control the gas output of the gas bombe 410 by adjusting the degree of opening and closing. The flow controller 233 may control the flow rate of gas to supply gas to the handpiece 100.

The control unit 240 may control the overall system operation of the components of the plasma treatment device 1000. The control unit 240 may process input signals, output signals, data, information, and the like through the components, or run applications stored in the memory, to provide and process appropriate information and functions for the user. The control unit 240 may receive a direct current voltage of 24 V through the converter 220 to operate and communicate with the pressure sensor 231, the electronic valve 232, and the flow controller 233 of the pneumatic unit 230, to control the flow rate and output of the supplied gas. In addition, the control unit 240 may drive its own set programs to provide visual information and an input window to the monitor 250 described later. In addition, the control unit 240 may apply a direct current voltage of 10 V to the handpiece 100 through the hybrid cable 300 described later.

The monitor 250 may be located on top of the main body 200 and may display medical information. When power is supplied to the main body 200, a treatment program may be driven and the monitor 250 may display the medical information. In this case, the displayed medical information may be various medical information, such as patient information, treatment time, treatment settings, current voltage, gas flow rate, device abnormalities, other information, and the like. In addition, the monitor 250 may input operation commands for operating the plasma treatment device 1000, and may input various settings, such as power operation, treatment time settings, gas flow rate settings, power frequency settings, to perform operations of the plasma treatment device 1000. The monitor 250 may be capable of inputting operational commands by various input means, such as buttons, switches, levers, touch screens, and keyboards, and may preferably be made with a touch screen.

The main body 200 may have a ventilation opening 260 formed on one side. The ventilation opening 260 may dissipate heat inside the main body 200 to prevent failure and safety accidents due to overheating.

The hybrid cable 300 according to the present invention may connect the handpiece 100 and the main body 200. The hybrid cable 300 may simultaneously supply voltage and gas from the main body 200 to the handpiece 100. Here, since a low voltage of 10 V direct current is supplied to the handpiece 100, high voltage does not flow in the hybrid cable 300 to improve safety, and manufacturing the cable is not complex, and less noise may be generated.

The plasma treatment device 1000 according to the present invention may include a gas supply unit 400 for supplying gas. The gas supply unit 400 may include the gas bombe 410, a gas tube 420, and a gas socket (not illustrated).

The gas bombe 410 may provide a space for storing gas. The gas may be inert gas, such as argon gas or nitrogen gas, and preferably, argon gas may be used.

The gas tube 420 may transport gas stored in the gas bombe 410 to the main body 200.

The gas socket (not illustrated) may connect the gas tube 420 and the main body 200 to supply gas to the plasma treatment device 1000. The main body 200 may have a gas socket groove 270 formed on one side, so that the gas socket (not illustrated) may be connected to the main body 200.

Here, the gas may be controlled in flow rate and output by the pneumatic unit 230. Preferably, the argon gas may be controlled at a flow rate of 4 l/m to 5 l/m. When the argon gas is supplied at a flow rate of less than 4 l/m or greater than 5 l/m, the generation of ozone that is harmful gas produced during plasma generation increases, which may reduce stability.

The plasma treatment device 1000 according to the present invention may have an excellent effect on skin regeneration treatment and skin beauty, such as atopic treatment, wound treatment, burn treatment, wrinkle improvement, and skin beauty, by suppressing an overactive immune response. In particular, the plasma treatment device 1000 according to the present invention inhibits the differentiation of Th2 cell that is the main cause of atopic dermatitis, and inhibits the infiltration of mast cells and eosinophils into skin tissue to have an excellent effect in atopic treatment.

The amount of ozone generated during the plasma generation of Examples 1 to 4 below was measured by varying the handpiece applied voltage and argon gas flow conditions of the plasma treatment device according to the present invention. This identified desirable handpiece applied voltage and argon gas flow conditions under which the amount of ozone generated from the plasma treatment device could be suppressed. Here, the amount of ozone generated of the plasma treatment device was measured by placing the handpiece in an upright position to generate plasma and measuring the amount of ozone generated per minute from the side of the handpiece.

### Example 1

The amount of ozone generated was measured for 10 minutes under the following conditions: handpiece applied voltage 9 V, argon gas flow rate 4 l/m, and measurement distance 10 mm.

### Example 2

The amount of ozone generated was measured for 10 minutes under the following conditions: handpiece applied voltage 10 V, argon gas flow rate 4 l/m, and measurement distance 10 mm.

TABLE 1

| | | 1 | 2 | 3 | 4 | 5 | 6 | 7 | 8 | 19 | 10 |
|---|---|---|---|---|---|---|---|---|---|---|---|
| | | min | mins | mins | mins | mins | mins | mins | mins | mins | mins |
| Amount of | Example 1 | 0.007 | 0.006 | 0.005 | 0.006 | 0.004 | 0.003 | 0.006 | 0.007 | 0.006 | 0.006 |
| ozone | Example 2 | 0.013 | 0.010 | 0.011 | 0.008 | 0.007 | 0.007 | 0.006 | 0.007 | 0.006 | 0.006 |
| generated | | | | | | | | | | | |
| (ppm) | | | | | | | | | | | |

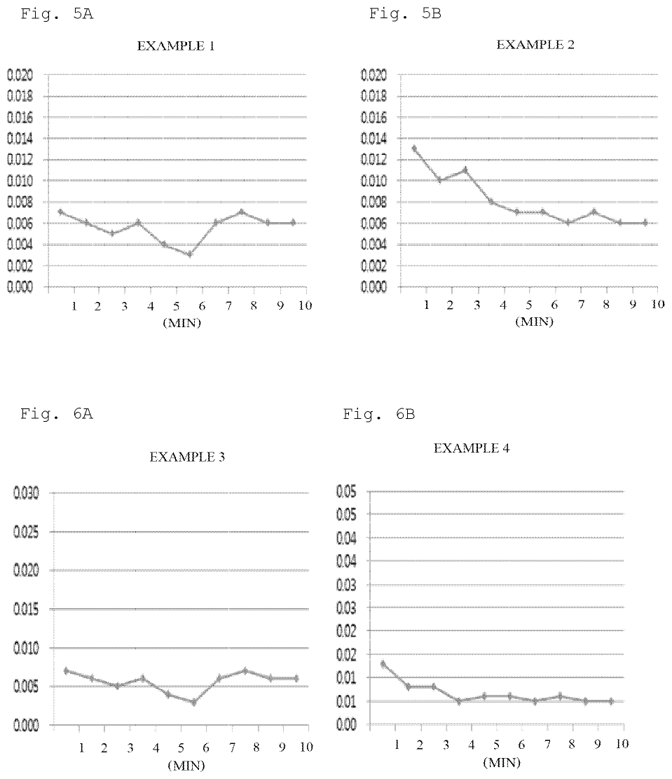

FIGS. 5A and 5B are graphs illustrating the amount of ozone generated of Example 1 and Example 2 according to applied voltage of the plasma treatment device. As a result of the measurement of the amount of ozone generated of the plasma treatment device according to the voltage, the amount of ozone generated after 10 minutes was 0.006 ppm in both Example 1 and Example 2. However, it can be seen that when a direct current voltage of 9 V is applied in Example 1, the initial amount of ozone generated is less than when a direct current voltage of 10 V is applied in Example 2. Therefore, preferably, when plasma is generated by setting the handpiece applied voltage to 9V, less ozone may be generated.

### Example 3

The amount of ozone generated was measured for 10 minutes under the following conditions: handpiece applied voltage 9 V, argon gas flow rate 4 l/m, and measurement distance 10 mm.

### Example 4

The amount of ozone generated was measured for 10 minutes under the following conditions: handpiece applied voltage 9 V, argon gas flow rate 5 l/m, and measurement distance 10 mm.

TABLE 2

| | | 1 | 2 | 3 | 4 | 5 | 6 | 7 | 8 | 9 | 10 |
|---|---|---|---|---|---|---|---|---|---|---|---|
| | | min | mins | mins | mins | mins | mins | mins | mins | mins | mins |
| Amount of | Example 3 | 0.007 | 0.006 | 0.005 | 0.006 | 0.004 | 0.003 | 0.006 | 0.007 | 0.006 | 0.006 |
| ozone | Example 4 | 0.013 | 0.008 | 0.008 | 0.005 | 0.006 | 0.006 | 0.005 | 0.006 | 0.005 | 0.005 |
| generated | | | | | | | | | | | |
| (ppm) | | | | | | | | | | | |

FIGS. 6A and 6B are graphs illustrating the amount of ozone generated of Example 3 and Example 4 according to a flow rate of argon gas of the plasma treatment device. As a result of measuring the amount of ozone generated from the plasma treatment device according to the voltage, it can be seen that the amount of ozone generated of the argon gas flow rate of 5 l/m in Example 4 was high at the beginning, but the amount of ozone generated was less with 0.005 ppm after 10 minutes. Therefore, when plasma is generated by setting the flow rate of argon gas to 5 l/m, less ozone may be generated.

In Examples 5 and 6 below, the amount of ozone generated when plasma is generated by supplying the handpiece test product with an applied voltage of 9 V and a flow rate of 5 l/m of argon gas, which are preferred plasma generation conditions according to Examples 1 to 4 above, was measured.

### Example 5

The amount of ozone generated was measured for 10 minutes under the following conditions: handpiece applied voltage 9V, argon gas flow rate 5 l/m, and measurement distance 10 mm.

### Example 6

The amount of ozone generated was measured for 10 minutes under the following conditions: handpiece applied voltage 9V, argon gas flow rate 5 l/m, and measurement distance 10 mm.

TABLE 3

| | | 1 | 2 | 3 | 4 | 5 | 6 | 7 | 8 | 9 | 10 |
|---|---|---|---|---|---|---|---|---|---|---|---|
| | | min | mins | mins | mins | mins | mins | mins | mins | mins | mins |
| Amount of | Example 5 | 0.014 | 0.016 | 0.017 | 0.019 | 0.018 | 0.019 | 0.020 | 0.019 | 0.020 | 0.019 |
| ozone | Example 6 | 0.015 | 0.017 | 0.018 | 0.017 | 0.017 | 0.018 | 0.019 | 0.021 | 0.021 | 0.022 |
| generated | | | | | | | | | | | |
| (ppm) | | | | | | | | | | | |

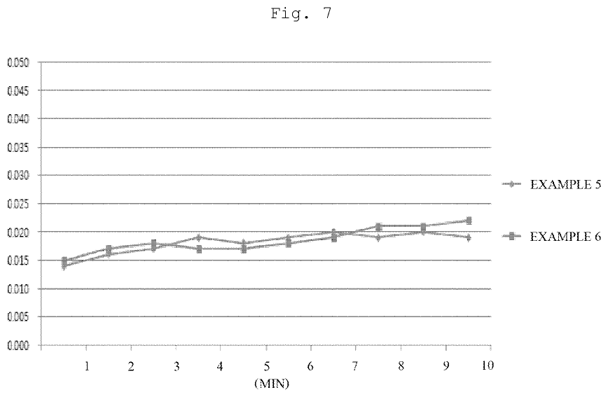

FIG. 7 is a graph of the amount of ozone generated of Example 5 and Example 6. After 10 minutes in Example 5, the amount of ozone generated was measured to be 0.019 ppm, and after 10 minutes in Example 6, the amount of ozone generated was measured to be 0.022 ppm. This confirms that both Example 5 and Example 6 met the ozone generation test safety standard of 0.05 ppm or less. In addition, as a result of the measurement of the ozone distribution for 10 minutes, the numerical values of the amounts of ozone generated measured for 10 minutes are similar, indicating that the handpiece is operating uniformly to generate plasma.

Although the present invention has been described with reference to the exemplary embodiments of the present invention, but it can be understood that those skilled in the art may variously modify and change the present invention without departing from the spirit and scope of the present invention described in the accompanying claims.

1. A plasma treatment device, comprising:
a handpiece for generating plasma and emitting the generated plasma onto skin;
a main body for supplying voltage and gas to the handpiece; and
a hybrid cable connecting the main body and the handpiece,
wherein the handpiece amplifies a low voltage to a high voltage to generate the plasma.

2. The plasma treatment device of claim 1, wherein the handpiece includes:
a plasma generating unit for generating the plasma by being supplied with the voltage and the gas;
a plasma output unit for emitting the plasma onto the skin; and
a handpiece tip positioned on a front surface of the plasma output unit to maintain a constant distance between the plasma output unit and the skin, and
the plasma is generated by amplifying a direct current voltage of 9 V to 10 V to an alternating current voltage of 700 V to 900 V at a frequency of 18 kHz to 20 kHz.

3. The plasma treatment device of claim 2, wherein the plasma generating unit includes a dielectric including one or more of quartz, sapphire, glass, ceramic, polymer film, and polyetherimide (PEI).

4. The plasma treatment device of claim 2, wherein the plasma output unit is formed with a nozzle which emits the plasma, and
the nozzle is at least one.

5. The plasma treatment device of claim 2, wherein the handpiece tip is replaceable.

6. The plasma treatment device of claim 1, wherein the main body includes:
a power unit for receiving an alternating current voltage;
a converter for converting the alternating current voltage to a direct current voltage;
a pneumatic unit for controlling the flow rate and output of the gas;
a control unit for controlling an overall system operation; and
a monitor for displaying medical information.

7. The plasma treatment device of claim 6, wherein the power unit is applied with an alternating current voltage of 220V, and
the converter converts the alternating current voltage to a direct current voltage of 24 V.

8. The plasma treatment device of claim 6, wherein the pneumatic unit includes:
a pressure sensor for measuring a pressure of the gas;
an electronic valve for controlling an output of the gas; and
a flow controller for controlling a flow rate of the gas.

9. The plasma treatment device of claim 1, further comprising:
a gas supply unit for supplying the gas,
wherein the gas supply unit includes:
a gas bombe for storing the gas;
a gas tube for transporting the gas; and
a gas socket connecting the main body and the gas tube, and
the gas is argon gas, and a flow rate of the gas is from 4 l/m to 5 l/m.

10. The plasma treatment device of claim 1, wherein the main body includes a ventilation opening for dissipating heat within the main body.
